(19) United States
(12) Patent Application Publication
(10) Pub. No.: US 2024/0293056 A1
(43) Pub. Date: Sep. 5, 2024

(54) Capillary Blood Collection Device

(71) Applicant: Becton, Dickinson and Company, Franklin Lakes, NJ (US)

(72) Inventors: Anthony V. Torris, Mercer Island, WA (US); Charles Peter Althoff, West Nyack, NY (US); Vlad Yakhnich, Park Ridge, NJ (US)

(21) Appl. No.: 18/573,484

(22) PCT Filed: Jun. 23, 2022
(86) PCT No.: PCT/US22/34650

§ 371 Date: Dec. 22, 2023

Related U.S. Application Data
(60) Provisional application No. 63/216,287, filed on Jun. 29, 2021.

(51) Int. Cl. A61B 5/15; A61B 5/151; B01L 9/06

(52) U.S. Cl. CPC A61B 5/150343; A61B 5/150022; A61B 5/150068; A61B 5/150412; A61B 5/150748; A61B 5/15117; A61B 5/1519; A61B 5/15194; B01L 9/06; B01L 2200/021; B01L 2300/021; B01L 2300/042

(57) ABSTRACT

A device for obtaining a blood sample may include a collection container defining a cavity to receive the blood sample; and an adaptor defining a cavity to receive the collection container, wherein the adaptor comprises retention features to orient the collection container into a desired position when inserted into the adaptor.

### CROSS-REFERENCE TO RELATED APPLICATION

The present application claims priority to U.S. Provisional Application Ser. No. 63/216,287, filed Jun. 29, 2021, entitled “Capillary Blood Collection Device”, the entire disclosure of which is hereby incorporated by reference in its' entirety.

### BACKGROUND OF THE INVENTION

### Field of the Invention

The present disclosure relates generally to a device for obtaining a biological sample. More particularly, the present disclosure relates to an integrated finger-based capillary blood collection device with the ability to lance and squeeze a finger, collect, stabilize, and dispense a blood sample in a controlled manner.

### Description of Related Art

Devices for obtaining and collecting biological samples, such as blood samples, are commonly used in the medical industry. One type of blood collection that is commonly done in the medial field is capillary blood collection which is often done to collect blood samples for testing. Certain diseases, such as diabetes, require that the patient's blood be tested on a regular basis to monitor, for example, the patient's blood sugar levels. Additionally, test kits, such as cholesterol test kits, often require a blood sample for analysis. The blood collection procedure usually involves pricking a finger or other suitable body part in order to obtain the blood sample. Typically, the amount of blood needed for such tests is relatively small and a small puncture wound or incision normally provides a sufficient amount of blood for these tests. Various types of lancet devices have been developed which are used for puncturing the skin of a patient to obtain a capillary blood sample from the patient.

Many different types of lancet devices are commercially available to hospitals, clinics, doctors' offices, and the like, as well as to individual consumers. Such devices typically include a sharp-pointed member such as a needle, or a sharp-edged member such as a blade, that is used to make a quick puncture wound or incision in the patient's skin in order to provide a small outflow of blood. It is often physiologically and psychologically difficult for many people to prick their own finger with a hand-held needle or blade. As a result, lancet devices have evolved into automatic devices that puncture or cut the skin of the patient upon the actuation of a triggering mechanism. In some devices, the needle or blade is kept in a standby position until it is triggered by the user, who may be a medical professional in charge of drawing blood from the patient, or the patient himself or herself. Upon triggering, the needle or blade punctures or cuts the skin of the patient, for example, on the finger. Often, a spring is incorporated into the device to provide the “automatic” force necessary to puncture or cut the skin of the patient.

One type of contact activated lancet device that features automatic ejection and retraction of the puncturing or cutting element from and into the device is U.S. Pat. No. 9,380,975, which is owned by Becton, Dickinson and Company, the assignee of the present application. This lancet device includes a housing and a lancet structure having a puncturing element. The lancet structure is disposed within the housing and adapted for movement between a retaining or pre-actuated position wherein the puncturing element is retained within the housing, and a puncturing position wherein the puncturing element extends through a forward end of the housing. The lancet device includes a drive spring disposed within the housing for biasing the lancet structure toward the puncturing position, and a retaining hub retaining the lancet structure in the retracted position against the bias of the drive spring. The retaining hub includes a pivotal lever in interference engagement with the lancet structure. An actuator within the housing pivots the lever, thereby moving the lancet structure toward the rearward end of the housing to at least partially compress the drive spring, and releases the lever from interference engagement with the lancet structure. The blood sample that is received is then collected and/or tested. This testing can be done by a Point-of-Care (POC) testing device or it can be collected and sent to a testing facility.

Currently, capillary blood collection workflow is a complex multi-step process requiring high skill level. The multi-step nature of this process introduces several variables that could cause sample quality issues such as hemolysis, inadequate sample stabilization, and micro-clots. The use of lancet devices for obtaining blood samples can result in several variables that effect the collection of the capillary blood sample, including, but not limited to, holding the lancet still during the testing, obtaining sufficient blood flow from the puncture site, adequately collecting the blood, preventing clotting, and the like. Some of the most common sources of process variability are: (1) inadequate lancing site cleaning and first drop removal which can potentially result in a contaminated sample; (2) inconsistent lancing location and depth which could potentially result in insufficient sample volume and a large fraction of interstitial fluid; (3) inconsistent squeezing technique and excessive pressure near the lancing site to promote blood extraction (e.g., blood milking) which could potentially result in a hemolyzed sample; (4) variable transfer interfaces and collection technique which could potentially result in a hemolyzed or contaminated sample; and (5) inadequate sample mixing with an anticoagulant which could potentially result in micro-clots.

Capillary collection blood draws are typically performed by health care workers either using their fingers to manually squeeze the tissue around the puncture site or by a device using vacuum pressure to pull blood from the site.

Manually squeezing the collection site is a highly technique dependent process that leads to very large variation in success rate and sample quality (as measured by hemolysis—blood cell rupture). Health care workers typically adjust the pressure and rate at which they squeeze to compensate for patient-dependent differences in blood flow. Squeezing harder helps blood flow more quickly but also increases hemolysis. The location of squeezing also varies between health care workers depending on personal preference, experience, and hand fatigue. Some workers may even perform a process called “milking” of fingers, where they apply pressure starting at the base of the finger and slide towards the tip of finger. This process is discouraged as leading to poor sample quality by domestic and international health organizations.

Vacuum-powered devices standardize the pressure and technique of blood flow, but are typically plagued by poor overall blood flow. The maximum pressure than can be applied is limited by the difference between atmospheric pressure and absolute vacuum (˜14 psi), and devices only operate at a fraction of absolute vacuum. For reference, grip strength of men and women range from 50-100 lbs. on average, illustrating why manual methods are instead affected by hemolysis rather than flow. Vacuum methods also apply consistent pressure, limiting the ability of the tissue to replenish with blood.

Thus, there is a need in the art for a device that has the ability to lance and squeeze the finger, collect the sample, stabilize the sample, and subsequently dispense the sample in a controlled manner. There is also a need in the art for a device that simplifies and streamlines the capillary blood collection by eliminating workflow variabilities which are typically associated with low sample quality including hemolysis and micro-clots. There is still a further need in the art for a closed system collection and transfer that eliminate blood exposure and device reuse. There is still a further need in the art for a device that: (1) introduces flexibility in the accommodation of different capillary blood collection and transfer container; (2) has the capability to generate high quality uniformly mixed/stabilized capillary blood samples; (3) has the capability to generate on-board plasma from capillary plasma samples; (4) has the capability to collect large capillary blood samples (>50-500 μL) at reduced pain; (5) contains a unique sample identifier that is paired with patient information at the time of collection; (6) has the capability to collect capillary blood and perform on-board diagnostics; and (7) has multiple collection ports to collect a blood sample into different containers having the same or different anticoagulants. There is a further need in the art for a capillary blood collection device that includes a standardized and controlled location of applied pressure, an applied pressure that is high enough for adequate blood flow but below hemolysis thresholds, a defined rhythmic application of pressure rather than consistent pressure to allow blood to replenish in the finger, increasing average blood flow rate, and a reduced user fatigue by lowering maximum applied force by the operator.

### SUMMARY OF THE INVENTION

The present disclosure is directed to a device for obtaining a biological sample, such as a capillary blood collection device, which meets the needs set forth above and has the ability to lance and squeeze the finger, collect the sample, stabilize the sample, and subsequently dispense the sample in a controlled manner. The device also simplifies and streamlines the capillary blood collection by eliminating workflow variabilities which are typically associated with low sample quality including hemolysis and micro-clots.

The present disclosure includes a self-contained and fully integrated finger-based capillary blood collection device with ability to lance, collect, and stabilize high volume capillary blood sample, e.g., up to or above 500 microliters. The device simplifies and streamlines high volume capillary blood collection by eliminating workflow steps and variabilities which are typically associated with low sample quality including hemolysis, micro-clots, and patient discomfort. The device comprises a retractable lancing mechanism that can lance the finger and an associated blood flow path which ensures attachment and transfer of the capillary blood from the pricked finger site to the collection container. The device also includes a holder that can be cyclically squeezed to stimulate, i.e., pump, blood flow out of the finger and also an anticoagulant deposited in the flow path or collection container to stabilize collected sample.

According to one design, the device can comprise discrete components such as a holder, a lancet, and a collection container. According to another design, the lancet and collection container can be integrated into one device which is then used with the holder. According to yet another design, the holder, lancet, and collection container can be integrated into a single system. Any of these designs are envisioned to be used as a self-standing disposable device and/or in association with an external power source for pain reduction control. The capillary blood collection device can serve as a platform for various capillary blood collection containers ranging from small tubes to capillary dispensers, as well as on-board plasma separation modules. This capability extends the product flexibility to various applications including dispensing to a Point-of-Care (POC) cartridge or to a small collection tube transfer which can be used in a centrifuge or an analytical instrument.

In one embodiment of the present disclosure, a device for obtaining a blood sample may include a collection container defining a cavity to receive the blood sample; and an adaptor defining a cavity to receive the collection container, wherein the adaptor comprises retention features to orient the collection container into a desired position when inserted into the adaptor.

In one embodiment of the present disclosure, the adaptor may include at least one protrusion extending from an end of the adaptor. The collection container may define at least one groove configured to receive the at least one protrusion of the adaptor. The adaptor may be made of a transparent material such that a barcode positioned on the collection container is viewed through the adaptor. The adaptor may be made of Methylmethacrylate Acrylonitrile Butadiene Styrene. The adaptor may include a height adjusting member in a bottom of the adaptor to raise a height of the collection container in the adaptor. An upper edge of a height adjusting member may include a sinusoidal cam surface to self-orient the collection container when inserted into the adaptor. The adaptor may include axial retention features and rotational retention features. The adaptor may include rotational retention features.

In one embodiment of the present disclosure, a device for obtaining a blood sample may include a holder for receiving a sample source, the holder having an actuation portion and a port; a collection container defining a cavity to receive the blood sample; and an adaptor defining a cavity to receive the collection container.

In one embodiment of the present disclosure, the adaptor may include at least one protrusion extending from an end of the adaptor. The collection container may define at least one groove configured to receive the at least one protrusion of the adaptor. The adaptor may be made of a transparent material such that a barcode positioned on the collection container is viewed through the adaptor. The adaptor may be made of Methylmethacrylate Acrylonitrile Butadiene Styrene. The adaptor may include a height adjusting member in a bottom of the adaptor to raise a height of the collection container in the adaptor. An upper edge of a height adjusting member may include a sinusoidal cam surface to self-orient the collection container when inserted into the adaptor. The adaptor may include axial retention features and rotational retention features. The adaptor may include rotational retention features. A lancet may be removably connected to the port of the holder to lance the sample source.

The present invention is also described in the following clauses:

Clause 1: A device for obtaining a blood sample, the device comprising: a collection container defining a cavity to receive the blood sample; and an adaptor defining a cavity to receive the collection container, wherein the adaptor comprises retention features to orient the collection container into a desired position when inserted into the adaptor.

Clause 2: The device of Clause 1, wherein the adaptor includes at least one protrusion extending from an end of the adaptor.

Clause 3: The device of Clause 2, wherein the collection container defines at least one groove configured to receive the at least one protrusion of the adaptor.

Clause 4: The device of any of Clauses 1-3, wherein the adaptor is made of a transparent material such that a barcode positioned on the collection container is viewed through the adaptor.

Clause 5: The device of Clause 4, wherein the adaptor is made of Methylmethacrylate Acrylonitrile Butadiene Styrene.

Clause 6: The device of any of Clauses 1-5, wherein the adaptor includes a height adjusting member in a bottom of the adaptor to raise a height of the collection container in the adaptor.

Clause 7: The device of any of Clauses 1-6, wherein an upper edge of a height adjusting member includes a sinusoidal cam surface to self-orient the collection container when inserted into the adaptor.

Clause 8: The device of any of Clauses 1-7, wherein the adaptor comprises axial retention features and rotational retention features.

Clause 9: The device of any of Clauses 1-8, wherein the adaptor comprises rotational retention features.

Clause 10: A device for obtaining a blood sample, the device comprising: a holder for receiving a sample source, the holder having an actuation portion and a port; a collection container defining a cavity to receive the blood sample; and an adaptor defining a cavity to receive the collection container.

Clause 11: The device of Clause 10, wherein the adaptor includes at least one protrusion extending from an end of the adaptor.

Clause 12: The device of Clause 11, wherein the collection container defines at least one groove configured to receive the at least one protrusion of the adaptor.

Clause 13: The device of any of Clauses 10-12, wherein the adaptor is made of a transparent material such that a barcode positioned on the collection container is viewed through the adaptor.

Clause 14: The device of Clause 13, wherein the adaptor is made of Methylmethacrylate Acrylonitrile Butadiene Styrene.

Clause 15: The device of any of Clauses 10-14, wherein the adaptor includes a height adjusting member in a bottom of the adaptor to raise a height of the collection container in the adaptor.

Clause 16: The device of any of Clauses 10-15, wherein an upper edge of a height adjusting member includes a sinusoidal cam surface to self-orient the collection container when inserted into the adaptor.

Clause 17: The device of any of Clauses 10-16, wherein the adaptor comprises axial retention features and rotational retention features.

Clause 18: The device of any of Clauses 10-17, wherein the adaptor comprises rotational retention features.

Clause 19: The device of any of Clauses 10-18, further comprising a lancet removably connected to the port of the holder to lance the sample source.

### BRIEF DESCRIPTION OF THE DRAWINGS

FIG. 1 is a perspective view of a holder in accordance with an embodiment of the present invention.

FIG. 2 is a cross-sectional view of a device for obtaining a blood sample from a patient's finger and a lancet in accordance with another embodiment of the present disclosure.

FIG. 3 is a perspective view of a device for obtaining a blood sample from a patient's finger and a collection container in accordance with another embodiment of the present disclosure.

FIG. 4 is a perspective view of a collection container received in an adaptor according to one embodiment of the present disclosure.

FIG. 5 is a perspective view of a collection container received in an adaptor according to another embodiment of the present disclosure.

FIG. 6 is another perspective view of the adaptor of FIG. 4.

FIG. 7 is another perspective view of the adaptor of FIG. 5.

FIG. 8 is a cross-sectional view of the collection container and the adaptor of FIG. 4.

FIG. 9 is a cross-sectional view of the collection container and the adaptor of FIG. 5.

FIG. 10 is a schematic illustration of a use of a tool to remove a cap of a collection container held in the adaptor of FIG. 5.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

The present disclosure is directed to a device for obtaining a biological sample, such as a capillary blood collection device, which meets the needs set forth above and has the ability to lance and squeeze the finger, collect the sample, stabilize the sample, and subsequently dispense the sample in a controlled manner. The device also simplifies and streamlines the capillary blood collection by eliminating workflow variabilities which are typically associated with low sample quality including hemolysis and micro-clots. The device may be used by healthcare professionals, such as doctors and nurses, or patients that use a self-application of the device.

Blood collection is fundamentally driven by pressure-driven flow. Devices or techniques either reduce the pressure outside the blood vessel (vacuum-powered flow) or increase the pressure inside the vessels. Both approaches increase the difference between the blood vessel pressure and external pressure, and increase the flow rate from inside the vessel to outside where the collection container is present. The location of squeezing can also be critical, as soft tissues (e.g. fat, skin, and musculature) are perfused with blood while hard tissues and joints are poorly perfused or are too mechanically stable to compress without patient pain.

Red blood cells (RBCs) are subject to hemolysis during collection. Hemolysis (RBC destruction) contaminates samples for diagnostic analysis, both by spilling cell contents into the liquid serum of the sample and by coloring the serum red via hemoglobin and interfering with colorimetric reactions. The amount of hemolysis during collection is driven by shear-mediated destruction of the cells due to flow rate and flow path as well as pressure-driven hemolysis where physical compression of tissues and vessels can damage cells. Hemolysis can therefore be controlled by ensuring that applied pressures and flows are not too high in any of the locations of the finger being squeezed.

The present disclosure includes a self-contained and fully integrated finger-based capillary blood collection device with ability to lance, collect, and stabilize high volume capillary blood sample, e.g., up to or above 500 microliters. The device simplifies and streamlines high volume capillary blood collection by eliminating workflow steps and variabilities which are typically associated with low sample quality including hemolysis, micro-clots, and patient discomfort. The device comprises a retractable lancing mechanism that can lance the finger and an associated blood flow path which ensures attachment and transfer of the capillary blood from the pricked finger site to the collection container. The device also includes a holder that can be cyclically squeezed to stimulate, i.e., pump, blood flow out of the finger and also an anticoagulant deposited in the flow path or collection container to stabilize collected sample.

According to one design, the device can comprise discrete components such as a holder, a lancet, and a collection container. According to another design, the lancet and collection container can be integrated into one device which is then used with the holder. According to yet another design, the holder, lancet, and collection container can be integrated into a single system. Any of these designs are envisioned to be used as a self-standing disposable device and/or in association with an external power source for pain reduction control. The capillary blood collection device can serve as a platform for various capillary blood collection containers ranging from small tubes to capillary dispensers, as well as on-board plasma separation modules. This capability extends the product flexibility to various applications including dispensing to a Point-of-Care (POC) cartridge or to a small collection tube transfer which can be used in a centrifuge or an analytical instrument.

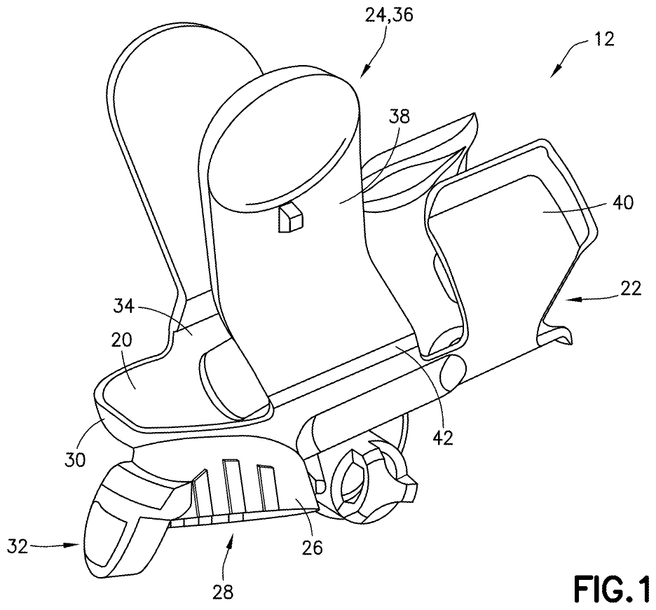

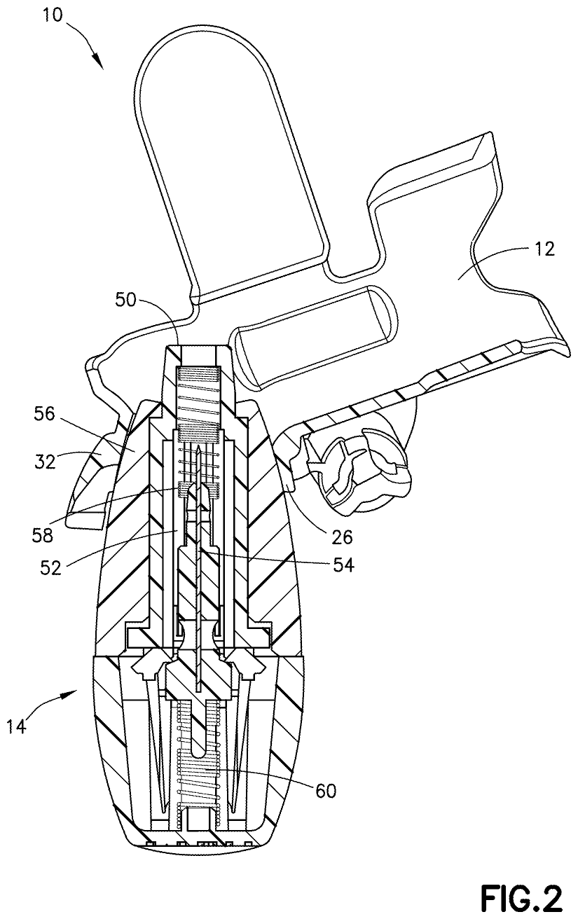

Referring to FIGS. 1-3, in an exemplary embodiment, a device 10 of the present disclosure includes discrete components, e.g., a holder 12 (as shown in FIG. 1), a lancet housing or lancet 14 (as shown in FIG. 2), and a collection container 16. In another exemplary embodiment, a semi-integrated device of the present disclosure may include an at-angle flow and include an integrated lancet housing and collection container which can be connected with a separate holder. In another exemplary embodiment, a semi-integrated device of the present disclosure may have an in-line flow and include an integrated lancet housing and collection container which can be connected with a separate holder. In another exemplary embodiment, an integrated device of the present disclosure may have an at-angle flow and include an integrated holder, lancet housing, and collection container. In another exemplary embodiment, an integrated device of the present disclosure may have an in-line flow and include an integrated holder, lancet housing, and collection container.

Referring to FIG. 1, an exemplary embodiment of a holder 12 of the present disclosure that is able to receive a sample source, e.g., a finger 19, for supplying a biological sample, such as a blood sample 18, is shown and described. A holder 12 of the present disclosure generally includes a finger receiving portion 20 having a first opening 22 (FIG. 1), an actuation portion 24, a port 26 having a second opening 28, and a finger end guard 30. In one embodiment, the finger end guard 30 provides a stop portion for properly aligning and securing a finger 19 within the holder 12. The finger end guard 30 further assists in ensuring the patient's finger 19 is placed at a proper position within the finger receiving portion 20 so that applied pressure to the patient's finger 19 will result in adequate blood flow.

The first opening 22 of the finger receiving portion 20 is configured for receiving a sample source, e.g., a finger 19, for supplying a biological sample, such as a blood sample 18. It can be appreciated that the sample source could include other parts of the body capable of fitting within the first opening 22. The port 26 is in communication with the finger receiving portion 20. For example, with a finger 19 received within the holder 12, the port 26 is in communication with a portion of the finger 19. A holder 12 of the present disclosure can be sized to accommodate all finger sizes.

The second opening 28 of the port 26 is configured for receiving a lancet housing 14 and a collection container 16 as described in more detail below. In one embodiment, the port 26 includes a locking portion 32 for securely receiving the lancet housing 14 and the collection container 16 within the port 26.

In one embodiment, the actuation portion 24 is transitionable between a first position in which the holder 12 defines a first diameter and a second position which the holder 12 defines a second diameter, wherein the second diameter is less than the first diameter. In one embodiment, the actuation portion 24 is transitionable between a first position in which the holder 12 defines a first elliptical shape, and a second position in which the holder 12 defines a second elliptical shape, wherein the first elliptical shape is different than the second elliptical shape. In this manner, with the holder 12 in the second position with a reduced diameter, a portion of the holder 12 contacts the sample source and the actuation portion 24 of the holder 12 is able to pump and/or extract blood 18 as described in more detail below.

Referring to FIG. 1, in one embodiment, the actuation portion 24 includes a contact member 34. With the actuation portion 24 in the first position, the contact member 34 is in a disengaged position, i.e., the contact member 34 is provided in a first position with respect to a sample source, e.g., the finger 19, such that the contact member 34 may be in slight contact therewith. With the actuation portion 24 in the second position, the contact member 34 is in an engaged position, i.e., the contact member 34 is provided in a second position with respect to the sample source, e.g., the finger 19, such that the contact member 34 is in an applied pressure contact with the finger 19, and the actuation portion 24 of the holder 12 is able to pump and/or extract blood 18. For example, with the contact member 34 in the engaged position, the contact member 34 exerts a pressure on the sample source.

Referring to FIG. 1, in one embodiment, the actuation portion 24 includes a pumping member 36 for applying pressure to the sample source, e.g., the finger 19. In one embodiment, the pumping member 36 comprises a pair of opposed tabs or wings 38. In such an embodiment, each tab 38 may include a contact member 34. In one embodiment, the holder 12 includes a living hinge portion 42. The living hinge portion 42 allows a user to squeeze the wings 38 between a first position (passive state) and a second position (active state). The use of the tabs or wings 38 to draw blood 18 out of a patient's finger 19 minimizes hemolysis while maintaining an adequate flow of blood from the patient's finger 19. A resting position and hinge of the wings 38 are designed to maintain contact and retention with the smallest patient finger that can fit into a holder 12 while flexing to accommodate the largest patient finger within a holder 12 without blood occlusion.

Advantageously, the holder 12 of the present disclosure allows a user to repeatedly squeeze and release the wings 38 to pump and/or extract blood 18 from a finger 19 until a desired amount of blood 18 is filled in a collection container 16. The wings 38 are configured to flex to maintain gentle contact with a range of patient finger sizes that may be used with the holder 12 and to retain the holder 12 on the patient's finger 19.

Advantageously, with the holder 12 placed onto a finger 19, the holder 12 does not constrict the blood flow and defines lancing and finger squeezing locations. The squeezing tabs or wings 38 provide a pre-defined range of squeezing pressure that is consistently applied throughout a finger 19. By doing so, the holder 12 provides a gentle controlled finger massage that stimulates blood extraction and minimizes any potential hemolysis.

Referring to FIG. 1, in one embodiment, the holder 12 includes a stability extension portion 40. This provides additional support for the holder 12 to be securely placed onto a finger 19. In one embodiment, the finger receiving portion 20 forms a generally C-shaped member and includes a plurality of inner gripping members for providing additional grip and support for the holder 12 to be securely placed onto a finger 19. The stability extension portion 40 assists in maintaining contact with the patient's finger 19 during use of the holder 12 while avoiding the blood supply and knuckles of the patient's finger 19.

In one embodiment, the finger receiving portion 20 is formed of a flexible material. In some embodiments, the finger receiving portion 20 and the port 26 are formed from a flexible material.

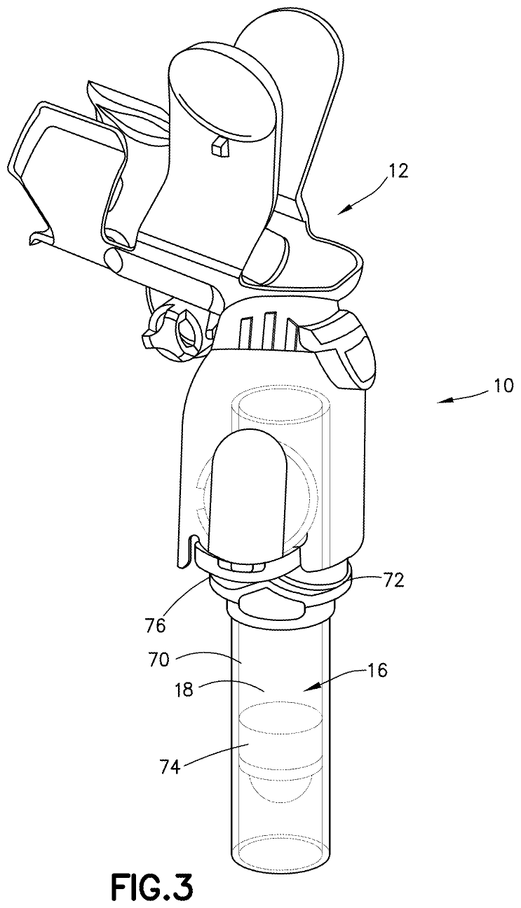

A device 10 for obtaining a blood sample 18 (shown in FIG. 3) of the present disclosure includes a lancet housing or lancet 14 that is removably connectable to a port 26 of a holder 12. Referring to FIG. 2, in one embodiment, the lancet housing 14 includes an inlet or opening 50, an interior 52, a puncturing element 54, an engagement portion 56, a retractable mechanism 58, and a drive spring 60. In one embodiment, the puncturing element 54 is moveable between a pre-actuated position wherein the puncturing element 54 is retained within the interior 52 of the lancet housing 14 and a puncturing position wherein at least a portion of the puncturing element 54 extends through the inlet 50 of the lancet housing 14 to lance a portion of a finger 19.

In one embodiment, the lancet 14 of the present disclosure is a contact activated lancet and may be constructed in accordance with the features disclosed in U.S. Patent Application Publication No. 2006/0052809 filed May 6, 2005, entitled “Contact Activated Lancet Device”, and commonly assigned with the present application, the entire disclosure of which is hereby expressly incorporated herein by reference thereto.

In one embodiment, the lancet housing 14 may be a separate component from the holder 12 and the collection container 16. In some embodiments, the collection container 16 and the lancet housing 14 form a single component that is removably connectable to the port 26 of the holder 12. In some embodiments, the collection container 16, the lancet housing 14, and the holder 12 form a single component.

Referring to FIG. 2, in one embodiment, with the holder 12 and the lancet housing 14 being separate components, the lancet housing 14 is removably connectable to the port 26 of the holder 12. In such an embodiment, the lancet housing 14 includes an engagement portion 56. Referring to FIG. 2, in one embodiment, the lancet housing 14 is pushed into the port 26 of the holder 12 such that the engagement portion 56 of the lancet housing 14 is locked within the locking portion 32 of the holder 12. In this manner, the lancet housing 14 is securely connected and locked to the holder 12 such that the puncturing element 54 of the lancet housing 14 can be activated to lance or puncture a sample source, e.g., a finger 19. In some embodiments, the port 26 of the holder 12 includes a plurality of ribs for securing and locking the lancet 14 or the collection container 16 in the port 26.

To activate the lancet 14, the lancet 14 is pushed against a finger 19 to activate a retractable mechanism 58 of the lancet 14 to lance a finger 19. The lancet 14 of the present disclosure consistently delivers correct lancing depth and a pre-defined lancing location, thus ensuring a sufficient sample volume.

In one embodiment, the lancet 14 includes a drive spring 60 disposed within the interior 52 of the lancet housing 14 for biasing the puncturing element 54 toward the puncturing position. After puncturing, the puncturing element 54 is immediately retracted and safely secured within the interior 52 of the lancet housing 14.

In one embodiment, the lancet 14 of the present disclosure is used to lance the skin of a finger 19 and then a blood sample 18 is squeezed into a collection container 16 as described in more detail below.

In one embodiment, the lancet housing 14 of the present disclosure is used to lance the skin of a finger 19 along a lance path and then a blood sample 18 flows down a blood flow path at an angle to the lance path as described in more detail below.

In one embodiment, the lancet 14 includes a hollow needle. In such an embodiment, the lancet housing 14 of the present disclosure is used to lance the skin of a finger 19 along a lance path and then a blood sample 18 flows along a parallel blood flow path through the hollow needle.

As shown in FIG. 3, a device 10 for obtaining a blood sample 18 of the present disclosure includes a collection container 16 that is removably connectable to the port 26 of the holder 12. The collection container 16 defines a collection cavity 70 for receiving a blood sample 18, a container engagement portion 72, a blood collector portion 74, and a cap or septum 76. Once a desired amount of blood 18 is collected within the container 16, a blood collector portion 74 is detached from the collection device 10 in order to send a collected sample 18 to a diagnostic instrument and/or testing device. The blood collector portion 74 is sealed via the cap or septum 76 once removed from the collection device 10 to protectively seal the blood sample 18 within the collection cavity 70.

In one embodiment, the collection container 16 may be a separate component from the holder 12 and the lancet housing 14. In some embodiments, the collection container 16 and the lancet housing 14 form a single component that is removably connectable to the port 26 of the holder 12. In some embodiments, the collection container 16, the lancet housing 14, and the holder 12 form a single component.

In one embodiment, with the holder 12 and the collection container 16 being separate components, the container 16 is removably connectable to the port 26 of the holder 12. In such an embodiment, the container 16 includes a container engagement portion 72. In one embodiment, the container 16 is pushed into the port 26 of the holder 12 such that the container engagement portion 72 of the container 16 is locked within the locking portion 32 of the holder 12. In this manner, the container 16 is securely connected and locked to the holder 12 such that a blood sample 18 can safely flow from the finger 19 within the holder 12 to the collection cavity 70 of the container 16.

It can be appreciated that several types of collection containers 16 can be used with the device 10 of the present disclosure. It can also be appreciated that the collection container 16 can be associated with a separate dispensing unit or the collection container 16 can include an integral dispensing portion for dispensing the blood 18 to a testing device.

Referring to FIG. 1, use of a device 10 of the present disclosure having discrete components, e.g., a holder 12, a lancet housing or lancet 14, and a collection container 16, will now be described.

Referring to FIG. 1, first a desired finger 19 is cleaned and a holder 12 having an appropriate size for the desired finger 19 is selected and placed onto the finger 19 securely. Next, referring to FIG. 2, a lancet housing 14 is connected to the port 26 of the holder 12. As discussed above, the lancet housing 14 is pushed into the port 26 of the holder 12 such that the engagement portion 56 of the lancet housing 14 is locked within the locking portion 32 of the holder 12. In this manner, the lancet housing 14 is securely connected and locked to the holder 12 such that the puncturing element 54 (FIG. 2) of the lancet housing 14 can be activated to lance or puncture a sample source, e.g., a finger 19. With the lancet 14 connected to the port 26 of the holder 12, the lancet 14 is in communication with the finger 19.

When it is desired to activate the lancet 14 to lance the skin of a finger 19, the lancet 14 is pushed against a finger 19 to activate a retractable mechanism 58 (FIG. 2) of the lancet 14 to lance a finger 19. The lancet 14 of the present disclosure consistently delivers correct lancing depth and a pre-defined lancing location, thus ensuring a sufficient sample volume.

After the finger 19 is lanced to create blood 18 flow from the finger 19, the lancet 14 is removed from the holder 12 and the collection container 16 is pushed into the port 26 of the holder 12. Referring again to FIG. 3, the container 16 is pushed into the port 26 of the holder 12 such that the container engagement portion 72 of the container 16 is locked within the locking portion 32 of the holder 12. In this manner, the container 16 is securely connected and locked to the holder 12 such that a blood sample 18 can safely flow from the finger 19 within the holder 12 to the collection cavity 70 of the container 16.

Referring to FIG. 1, with the container 16 properly secured to the holder 12 for collection of a blood sample 18, a user is able to repeatedly squeeze and release the wings 38 of the holder 12 to pump and/or extract blood 18 from a finger 19 until a desired amount of blood 18 is filled in a collection container 16. Advantageously, with the holder 12 placed onto a finger 19, the holder 12 does not constrict the blood flow and defines lancing and finger squeezing locations. The squeezing tabs or wings 38 provide a pre-defined range of squeezing pressure that is consistently applied throughout a finger 19. By doing so, the holder 12 provides a gentle controlled finger 19 massage that stimulates blood extraction and minimizes any potential hemolysis.

For example, referring to FIG. 1, in one embodiment, the actuation portion 24 includes a contact member 34. With the actuation portion 24 in the first position, the contact member 34 is in a disengaged position, i.e., the contact member 34 is in the first position with respect to the sample source, e.g., the finger 19. With the actuation portion 24 in the second position, the contact member 34 is in an engaged position, i.e., the contact member 34 is in the second position and in applied pressure contact with a sample source, e.g., the finger 19, and the actuation portion 24 of the holder 12 is able to pump and/or extract blood 18. For example, with the contact member 34 in the engaged position, the contact member 34 exerts a pressure on the sample source.

Once a desired amount of blood 18 is collected within the container 16, a blood collector portion 74 is detached from the collection device 10 in order to send a collected sample 18 to a diagnostic instrument and/or testing device. The blood collector portion 74 is scaled via the cap or septum 76 once removed from the collection device 10 to protectively seal the blood sample 18 within the collection cavity 70.

The devices of the present disclosure are compatible with any known testing device, whether the testing device is off-site or a point-of-care testing device. Various point-of-care testing devices are known in the art. Such point-of-care testing devices include test strips, glass slides, diagnostic cartridges, or other testing devices for testing and analysis. Test strips, glass slides, and diagnostic cartridges are point-of-care testing devices that receive a blood sample and test that blood for one or more physiological and biochemical states. There are many point-of-care devices that use cartridge based architecture to analyze very small amounts of blood bedside without the need to send the sample to a lab for analysis. This saves time in getting results over the long run, but creates a different set of challenges versus the highly routine lab environment. Examples of such testing cartridges include the i-STAT® testing cartridge from the Abbot group of companies. Testing cartridges such as the i-STAT® cartridges may be used to test for a variety of conditions including the presence of chemicals and electrolytes, hematology, blood gas concentrations, coagulation, or cardiac markers. The results of tests using such cartridges are quickly provided to the clinician.

The collection container 16 may also contain a sample stabilizer, e.g., an anticoagulant, to stabilize a blood sample 18 and/or a component of a blood sample 18 disposed therein. The collection container 16 may also include at least one fill line(s) corresponding to a predetermined volume of sample. The collection container may also indicate/meter a collected volume of blood.

Any of the devices for obtaining a blood sample of the present disclosure can be used as a self-standing disposable device and/or in association with an external power source for pain reduction control. For example, a portion of holder 12 may include embedded electrodes which receive a signal from an external pain control module to deliver at least one of heat, vibration, or transcutaneous electrical nerve stimulation (TENS) for pain reduction control. The devices for obtaining a blood sample of the present disclosure may also include various options for on-board plasma separation. The devices for obtaining a blood sample of the present disclosure may also include a unique sample identifier that can be paired with patient information at the time of collection. The devices for obtaining a blood sample of the present disclosure may also include on-board diagnostic feedback at the time of collection. A device for obtaining a blood sample of the present disclosure may also allow for dual collection, e.g., the collection of two samples into two separate containers, using multiple collection ports which enable the collection of multiple samples from the same source and treating the samples with different sample stabilizers, such as anticoagulants.

A device for obtaining a blood sample of the present disclosure significantly simplifies and de-skills large volume capillary collection from a finger relative to the conventional capillary collection using lancet and capillary tube. The devices of the present disclosure eliminate blood exposure and prevents device reuse.

The devices for obtaining a blood sample of the present disclosure simplify, deskill, and streamline the collection process. This is all achieved by a self-contained closed system device which after it is placed onto a finger will provide lancing, blood extraction, stabilization, and containment functions, all in one unit.

The devices for obtaining a blood sample of the present disclosure may be associated with a self-standing unit that provides automated pumping, controlled finger squeezing, and automated sample labeling and processing.

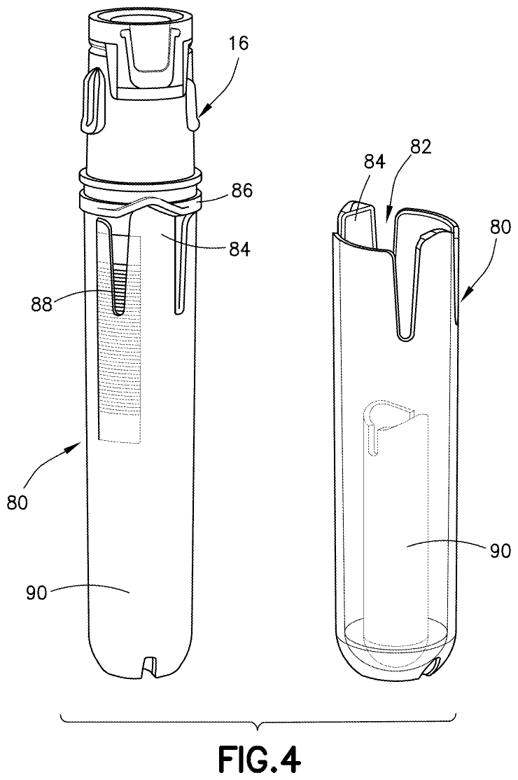

With reference to FIG. 4, according to one embodiment of the present disclosure, an adaptor 80 may be provided for use with the device 10. The adaptor 80 may be configured to receive the collection container 16 of the device 10 for automated processing on a blood analysis instrument or system (not illustrated). It is to be understood that the adaptor 80 may be configured for use with any known blood analysis instrument or system, such as a centrifuge. The device 10 may be designed to be functionally as small as possible for capillary blood collection from a patient's fingertip. A small, unobtrusive size for the device 10 is desired while the device 10 is connected to the patient's finger 19 during collection, and is desired to avoid interference with the patient's hand/wrist during lancing (pre-collection). The smaller size of the device 10, however, may not accommodate high-speed sample handling and automated processing on the blood analysis instrument or system. Therefore, the adaptor 80 may be utilized with the device 10 to solve this problem, thereby converting the device 10 to allow for high-speed sample handling and automated processing.

The adaptor 80 may define a cavity 82 that is configured to receive the collection container 16 of the device 10. The adaptor 80 may also include protrusions 84 that extend from an end of the adaptor 80. The protrusions 84 are configured to be received in a groove 86 defined on the collection container 16. The protrusions 84 may include a latch to snap into the groove 86 to secure the adaptor 80 to the collection container 16. In one embodiment, at least one of the protrusions 84 may extend further than the rest of the protrusions 84 so as to engage with the groove 86 to self-orient the collection container 16 within the adaptor 80. The collection container 16 may be rotated until the protrusion 84 slips into the groove 86, ensuring the collection container 16 is oriented in a desired position within the adaptor 80.

In one embodiment of the present disclosure, the adaptor 80 may be made of a transparent material that allows a user to scan a barcode 88 provided on the collection container 16 received in the adaptor 80 for patient/sample identification. The adaptor 80 may also include a height adjusting member 90 to elevate the blood sample 18 to an optimal height in the collection container 16 for each in-scope instrument. The adaptor 80 may include specific features for blood sample retention based on requirements for each device type. For an ethylenediaminetetraacetic acid (EDTA) device (shown in FIG. 4), the adaptor 80 provides axial retention for automatic cell resuspension. The adaptor 80 also provides rotational retention for barcode 88 scanning.

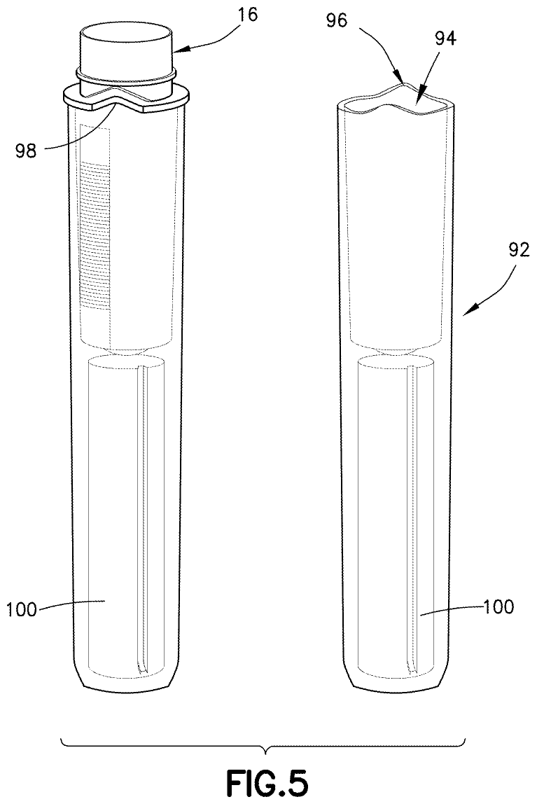

With reference to FIG. 5, in another embodiment of the present disclosure, a serum separating tube (SST) adaptor 92 is shown and described. The adaptor 92 is similar to the adaptor 80 described above but may include different dimensions to accept different types of collection containers 16. The adaptor 92 may define a cavity 94 that is configured to receive the collection container 16 of the device 10. The adaptor 92 may also include protrusions 96 that extend from an end of the adaptor 92. The protrusions 96 are configured to be received in a groove 98 defined on the collection container 16. The protrusions 96 may include a latch to snap into the groove 98 to secure the adaptor 92 to the collection container 16. In one embodiment, at least one of the protrusions 96 may extend further than the rest of the protrusions 96 so as to engage with the groove 98 to self-orient the collection container 16 within the adaptor 92. The collection container 16 may be rotated until the protrusion 96 slips into the groove 98, ensuring the collection container 16 is oriented in a desired position within the adaptor 92. The adaptor 92 may be configured to provide a robust rotational retention to aid in a de-capping operation. The adaptor 92 may also include a height adjusting member 100 to elevate the blood sample 18 to an optimal height in the collection container 16 for each in-scope instrument.

The adaptors 80, 92 are configured to accept the collection container 16 of the device 10 to change the outer diameter of the collection container 16. Effectively, the collection container 16 now has the outside diameter of the adaptor 80, 92. By increasing the outside diameter of the collection container 16, the collection container 16 effectively conforms to the dimensional needs of the fixturing and handling features of the blood analysis instrument or system. The adaptor 80, 92 assists in keeping the blood sample 18 centered with respect to the axis of the adaptor 80, 92.

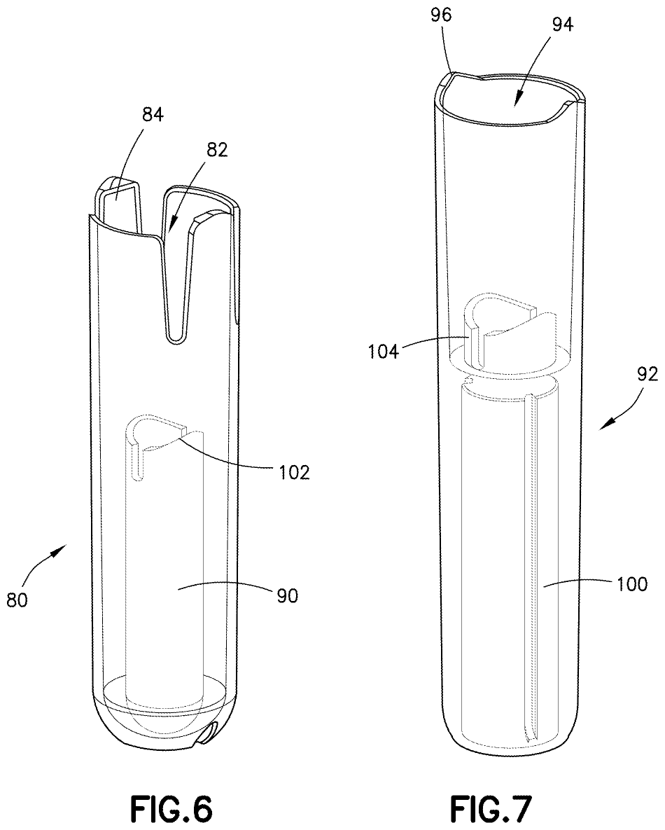

With reference to FIGS. 6 and 7, in further embodiments of the present disclosure, sinusoidal cam features 102, 104 provided on the height adjusting members 90, 100 of the adaptors 80, 92, respectively, may be provided to automatically orient the collection container 16 during insertion or installation of the collection container 16 into the adaptor 80, 92. A rib on the bottom surface of the collection container 16 may ride the top surface of the sinusoidal cam surfaces 102, 104 until the rib of the collection container 16 falls into a retention groove in the height adjusting member 90, 100. By providing the sinusoidal cam surfaces 102, 104 with the adaptors 80, 92, the collection container 16 can be inserted into the adaptor 80, 92 in any orientation and the sinusoidal cam surface 102, 104 will guide the collection container 16 into place, thereby orienting the alignment/retention features of the adaptor 80, 92 to the collection container 16. The sinusoidal cam surfaces 102, 104 may also be used to ensure the barcode label 88 is positioned optimally for scanning during automated processing of the collection container 16.

In some embodiments of the present disclosure, the adaptors 80, 92 may be made of optically clear material (such as Methylmethacrylate Acrylonitrile Butadiene Styrene (mABS)) to allow for barcode scanning through the adaptor 80, 92 sidewalls. By using such a material, the adaptor 80, 92 can be re-used between multiple collection containers 16 and blood samples 18, each with their own unique identifier being presented to the blood analysis instrument or system.

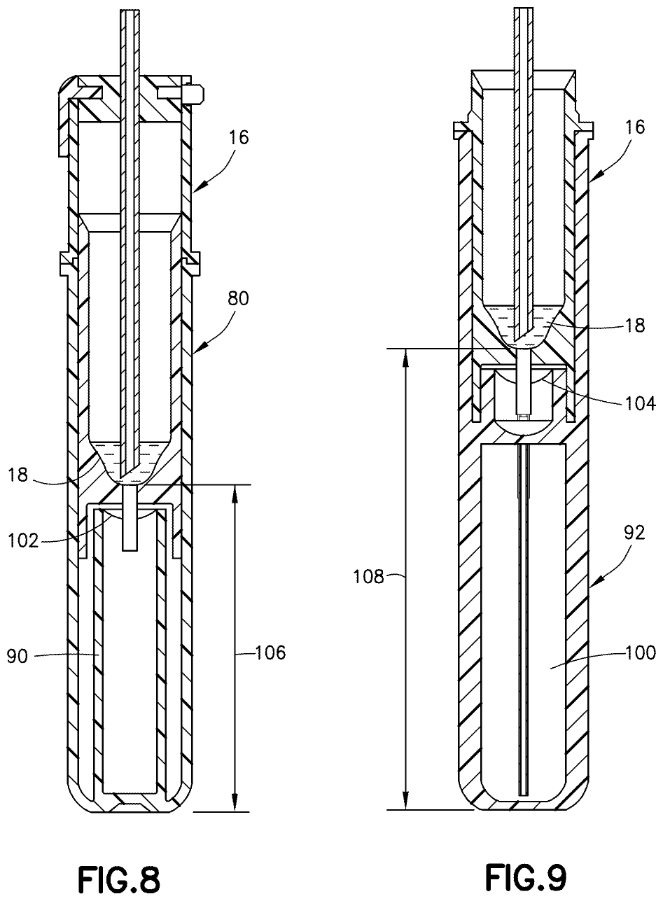

With reference to FIGS. 8 and 9, according to embodiments of the present disclosure, the adaptors 80, 92 may include the height adjusting members 90, 100 to raise the height of the blood sample 18 to an optimal height for scanning of the barcode 88 and to lift the bottom of cavity 70 of the collection container 16 (which holds the blood sample 18) to an optimal height for sample aspiration with minimum dead volume. The bottom offset 106, 108 of each respective adaptor 80, 92 is specific to each adaptor 80, 92 type (EDTA/SST).

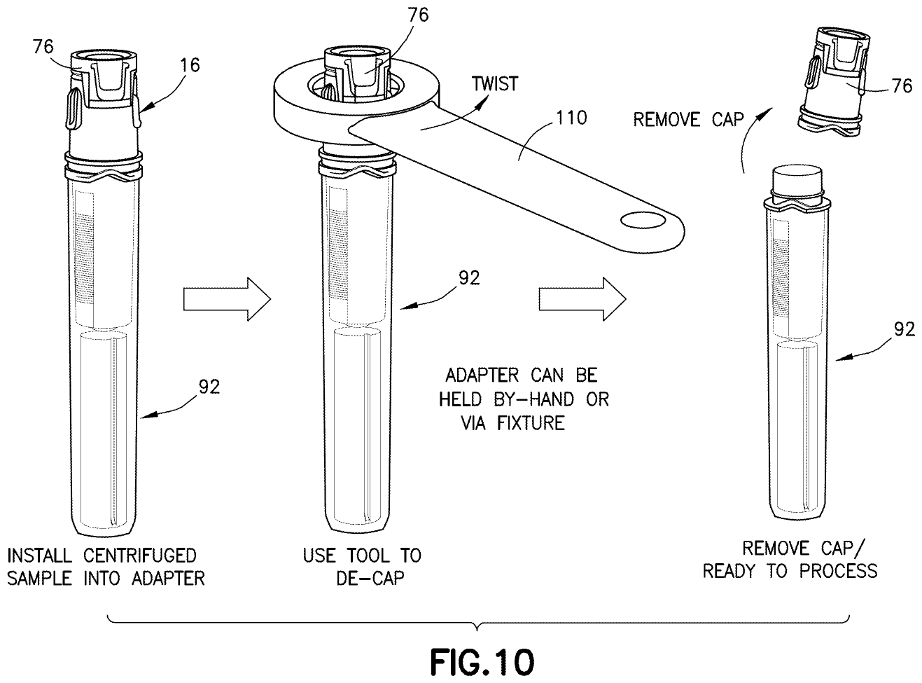

Each adaptor 80, 92 may include specific retention features to enable prep for and during automated sample processing with the blood analysis instrument or system. The adaptor 80 may include axial and rotational retention features. The axial retention features clip the collection container 16 into place and prevent disconnection from the adaptor 80 during automatic cell resuspension processes. The rotational retention features of the adaptor 80 allow the blood analysis instrument or system to spin the adaptor 80 and read the unique barcode 88 on the collection container 16. As shown in FIG. 10, in one embodiment of the present disclosure, the rotational retention features provided on the adaptor 92 rigidly hold the collection container 19 in place during a de-capping process using a tool 110. The tool 110 may be placed around the top end of the collection container 16 and twisted to remove the cap 76 from the collection container 16. Ribbed features in the false-bottom of the adaptor 92 allow for fixturing during the de-capping process, or the adaptor 92 may be held by hand during the de-capping operation.

The adaptors 80, 92, with their specific feature sets, convert the device 10 from being optimally sized for fingertip blood collection to being optimally sized, positioned, and restrained for automated processing and sampling via a blood analysis instrument or system. The adaptors 80, 92 allow for blood samples 18 from the device 10 to be processed using currently available blood analysis instruments and systems. Currently available adaptors, such as a microtainer adaptor, do not convert the outer diameter of the collection container 16 so that the collection container 16 can be handled during automated processing. Current adaptors do not include the features that allow for de-capping while installed in the adaptor 80, 92, nor do they elevate the barcode and bottom offset of the blood sample 18 to an optimal height. Further, no current adaptors feature sinusoidal cam surfaces to automatically orient the collection container 16 into the adaptor 80, 92.

While an embodiment of a capillary blood collection device is shown in the accompanying figures and described hereinabove in detail, other embodiments will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is:

1. A device for obtaining a blood sample, the device comprising:
a collection container defining a cavity to receive the blood sample; and
an adaptor defining a cavity to receive the collection container, wherein the adaptor comprises retention features to orient the collection container into a desired position when inserted into the adaptor.

2. The device of claim 1, wherein the adaptor includes at least one protrusion extending from an end of the adaptor.

3. The device of claim 2, wherein the collection container defines at least one groove configured to receive the at least one protrusion of the adaptor.

4. The device of claim 1, wherein the adaptor is made of a transparent material such that a barcode positioned on the collection container is viewed through the adaptor.

5. The device of claim 4, wherein the adaptor is made of Methylmethacrylate Acrylonitrile Butadiene Styrene.

6. The device of claim 1, wherein the adaptor includes a height adjusting member in a bottom of the adaptor to raise a height of the collection container in the adaptor.

7. The device of claim 1, wherein an upper edge of a height adjusting member includes a sinusoidal cam surface to self-orient the collection container when inserted into the adaptor.

8. The device of claim 1, wherein the adaptor comprises axial retention features and rotational retention features.

9. The device of claim 1, wherein the adaptor comprises rotational retention features.

10. A device for obtaining a blood sample, the device comprising:
a holder for receiving a sample source, the holder having an actuation portion and a port;
a collection container defining a cavity to receive the blood sample; and
an adaptor defining a cavity to receive the collection container.

11. The device of claim 10, wherein the adaptor includes at least one protrusion extending from an end of the adaptor.

12. The device of claim 11, wherein the collection container defines at least one groove configured to receive the at least one protrusion of the adaptor.

13. The device of claim 10, wherein the adaptor is made of a transparent material such that a barcode positioned on the collection container is viewed through the adaptor.

14. The device of claim 13, wherein the adaptor is made of Methylmethacrylate Acrylonitrile Butadiene Styrene.

15. The device of claim 10, wherein the adaptor includes a height adjusting member in a bottom of the adaptor to raise a height of the collection container in the adaptor.

16. The device of claim 10, wherein an upper edge of a height adjusting member includes a sinusoidal cam surface to self-orient the collection container when inserted into the adaptor.

17. The device of claim 10, wherein the adaptor comprises axial retention features and rotational retention features.

18. The device of claim 10, wherein the adaptor comprises rotational retention features.

19. The device of claim 10, further comprising a lancet removably connected to the port of the holder to lance the sample source.
